(19) United States
(12) Patent Application Publication
(10) Pub. No.: US 2017/0304171 A1
(43) Pub. Date: Oct. 26, 2017

(54) ALCOHOL-FREE EMULSION BASED ON NONIONIC SURFACTANTS, ON AN ANIONIC AMPHIPHILIC LIPID AND ON LIPOSOLUBLE ORGANIC UV-SCREENING AGENTS

(71) Applicant: L'OREAL, Paris (FR)

(72) Inventors: Fanny PLISMY-JUQUEL, Antony (FR); Sylvain HARRAULT, Paris (FR)

(21) Appl. No.: 15/523,510

(22) PCT Filed: Nov. 2, 2015
(86) PCT No.: PCT/EP2015/075472

§ 371 Date: May 1, 2017

(30) Foreign Application Priority Data
Nov. 3, 2014 (FR) 1460584

(51) Int. Cl. A61K 8/40; A61Q 17/04; A61K 8/92; A61K 8/37; A61K 8/06; A61K 8/44; A61K 8/39; A61Q 19/08; A61K 8/35

(52) U.S. Cl. CPC A61K 8/40; A61Q 19/08; A61Q 17/04; A61K 8/922; A61K 8/35; A61K 8/37; A61K 8/062; A61K 8/442; A61K 8/39; A61K 8/375; A61K 2800/21; A61K 2800/30; A61K 2800/596

(57) ABSTRACT

The present invention relates to an oil-in-water emulsion with globules of oil, the number-average size of which is less than or equal to 200.0 nm, comprising:
a) a continuous aqueous phase,
b) an oily phase dispersed in said aqueous phase,
c) a mixture of nonionic surfactants comprising:
   (i) at least one alkoxylated fatty ester, and
   (ii) at least one fatty ester of glycerol,
d) at least one anionic amphiphilic lipid,
e) at least one liposoluble organic UV-screening agent,
   characterized in that it does not comprise any monoalcohol comprising from 2 to 8 carbon atoms, and that the weight ratio of the amount of oily phase to the total amount of nonionic surfactants and anionic amphiphilic lipid is from 2 to 20.

The invention also relates to a cosmetic process for limiting the darkening of the skin and/or improving the colour and/or uniformity of the complexion, comprising the application, to the surface of the keratin material, of a composition as defined previously.

It also relates to a cosmetic process for preventing and/or treating the signs of ageing of a keratin material, comprising the application, to the surface of the keratin material, of a composition as defined previously.

It also relates to a cosmetic process for moisturizing a keratin material, comprising the application, to the surface of the keratin material, of a composition as defined previously.

The present invention relates to an oil-in-water emulsion with globules of oil, the number-average size of which is less than or equal to 200.0 nm, comprising:
a) a continuous aqueous phase,
b) an oily phase dispersed in said aqueous phase,
c) a mixture of nonionic surfactants comprising:
   (i) at least one alkoxylated fatty ester, and
   (ii) at least one fatty ester of glycerol,
d) at least one anionic amphiphilic lipid,
e) at least one liposoluble organic UV-screening agent,
   characterized in that it does not comprise any monoalcohol comprising from 2 to 8 carbon atoms, and that the weight ratio of the amount of oily phase to the total amount of nonionic surfactants and anionic amphiphilic lipid is from 2 to 20.

The invention also relates to the process for preparing said emulsion and to the uses thereof in cosmetic fields.

It also relates to a cosmetic process for limiting the darkening of the skin and/or improving the colour and/or uniformity of the complexion, comprising the application, to the surface of the keratin material, of a composition as defined previously.

It also relates to a cosmetic process for preventing and/or treating the signs of ageing of a keratin material, comprising the application, to the surface of the keratin material, of a composition as defined previously.

It also relates to a cosmetic process for moisturizing a keratin material, comprising the application, to the surface of the keratin material, of a composition as defined previously.

It is known that radiation with wavelengths of between 280 nm and 400 nm permits tanning of the human epidermis and that radiation with wavelengths of between 280 and 320 nm, known as UVB rays, harm the development of a natural tan. Exposure is also liable to induce impairment of the biomechanical properties of the epidermis, which is reflected by the appearance of wrinkles, leading to premature ageing of the skin.

It is also known that UVA rays with wavelengths of between 320 and 400 nm penetrate more deeply into the skin than UVB rays. UVA rays bring about immediate and persistent tanning of the skin. Under normal conditions, daily exposure to UVA rays, even of short duration, can result in damage to the collagen fibres and the elastin, which is reflected by a modification to the microrelief of the skin, the appearance of wrinkles and uneven pigmentation (liver spots, non-uniformity of the complexion).

Many cosmetic compositions for photoprotecting the skin (against UVA and/or UVB) have been proposed to date.

Anti-sun compositions are quite often in the form of an emulsion of oil-in-water type (i.e. a cosmetically acceptable support consisting of an aqueous, dispersing, continuous phase and of an oily, dispersed, discontinuous phase), which contains, in varying concentrations, one or more standard lipophilic and/or hydrophilic organic screening agents capable of selectively absorbing the harmful UV radiation, these screening agents (and the amounts thereof) being selected as a function of the desired sun protection factor, the sun protection factor (SPF) being expressed mathematically as the ratio of the dose of UV radiation required to reach the erythema-forming threshold with the UV-screening agent to the dose of the UV radiation required to reach the erythema-forming threshold without UV-screening agent.

Today, the majority of anti-sun products sold for daily photoprotection are in the form of an opaque emulsion or a more or less whitish emulsified gel. Due to the significant amount of screening agents to be introduced into the formulations, it is technically very difficult to propose thick transparent or translucent formulations with high protection factors which can be used in the context of daily photoprotection.

The transparent formulations currently on the market in the field of photoprotection are in the form of liquid or very slightly gelified lotions with a very high alcohol content, namely more than 50%, in order to guarantee a certain dissolution of the screening agents. Unfortunately, this type of highly alcoholic formulation cannot be used on all skin types and on the face as a care product.

Patent FR2924020 proposed an oil-in-water emulsion with globules of oil, the number-average size of which is less than or equal to 200 nm, comprising:
a) at least one surfactant chosen from ethoxylated fatty ethers, ethoxylated fatty esters, fatty esters of glycerol or mixtures thereof, and
b) at least one oil with a molecular weight of greater than 400 g/mol, in which the weight ratio of the amount of oily phase to the amount of solid surfactant ranges from 2 to 20, characterized in that it comprises at least one liposoluble organic UV-screening agent.

In order to obtain products which are stable on storage and transparent, these compositions generally contain at least one monoalcohol comprising from 2 to 8 carbon atoms and more particularly from 2 to 6 carbon atoms, such as ethanol, in amounts ranging up to 15% by weight relative to the total weight of these compositions.

During its extensive research, the applicant has observed that these formulations are not entirely satisfactory in terms of the photoprotective effectiveness, in particular in terms of the protection factors obtained. Moreover, these emulsions are prepared under restrictive conditions, by means of a high-pressure homogenizer at pressures of greater than or equal to 1000.0 bar.

There remains the need to produce formulations, based on UV-screening agents, which are transparent or translucent, stable on storage, with high protection factors, which can be used in the context of daily photoprotection (SPF>15) and which can be manufactured under conditions which are easier to implement, in particular with a homogenizer using pressures less than 1000.0 bar.

The applicant has discovered, surprisingly, that this objective can be achieved by using one or more liposoluble organic UV-screening agents in an emulsion free from monoalcohol comprising from 2 to 8 carbon atoms, comprising an oily phase dispersed in an aqueous phase and with globules of oil, the number-average size of which is less than or equal to 200.0 nm, comprising a mixture of specific nonionic surfactants and at least one anionic amphiphilic lipid, in which the weight ratio of the amount of oily phase to the total amount of nonionic surfactants and anionic amphiphilic lipid ranges from 2 to 20.

The emulsion obtained is stable on storage. It may contain significant amounts of oil while having good cosmetic properties and also satisfactory photoprotective effectiveness.

Moreover, the emulsion obtained has good moisturizing properties.

The present invention relates to an oil-in-water emulsion with globules of oil, the number-average size of which is less than or equal to 200.0 nm, comprising:

a) a continuous aqueous phase,

b) an oily phase dispersed in said aqueous phase,

c) a mixture of nonionic surfactants comprising:
(i) at least one alkoxylated fatty ester, and
(ii) at least one fatty ester of glycerol different from i),

d) at least one anionic amphiphilic lipid,

e) at least one liposoluble organic UV-screening agent,

characterized in that it does not comprise any monoalcohol comprising from 2 to 8 carbon atoms, and that the weight ratio of the amount of oily phase to the total amount of nonionic surfactants and anionic amphiphilic lipid(s) is from 2 to 20.

According to one particular form of the invention, the emulsion comprises a physiologically acceptable medium.

The invention also relates to the process for preparing said emulsion that will be defined in more detail below.

It also relates to a cosmetic process for limiting the darkening of the skin and/or improving the colour and/or uniformity of the complexion, comprising the application, to the surface of the skin, of a composition as defined previously.

It also relates to a cosmetic process for preventing and/or treating the signs of ageing of a keratin material, comprising the application, to the surface of the keratin material, of a composition as defined previously.

It also relates to a cosmetic process for moisturizing a keratin material, comprising the application, to the surface of the keratin material, of a composition as defined previously.

In the remainder of the present description, the term “liposoluble organic UV-screening agent” is intended to mean any organic compound for screening out UV radiation, which can be fully dissolved in molecular form or miscible in an oily phase of the emulsion or else can be dissolved in colloidal form (for example in micellar form) in an oily phase.

The term “emulsion not containing any monoalcohol comprising from 2 to 8 carbon atoms” is intended to mean any emulsion containing less than 0.1% by weight of monoalcohol comprising from 2 to 8 carbon atoms or even being completely devoid of monoalcohol comprising from 2 to 8 carbon atoms.

The term “monoalcohol comprising from 2 to 8 carbon atoms” is intended to mean any hydrocarbon-based (consisting of hydrogen and carbon atoms) organic compound comprising from 2 to 8 carbon atoms and just one alcohol function on the hydrocarbon-based chain.

In the context of the present invention, the term “keratin material” is intended to mean, in particular, the skin (body, face), the lips, the hair and the scalp. More particularly, the term “keratin material” is intended to mean the skin, the lips and the hair. When the cosmetic composition of the invention is a sun protection product, the term “keratin material” is preferably intended to mean the skin and the lips.

The term “physiologically acceptable” is intended to mean compatible with the skin and/or appendages thereof, which has a pleasant colour, odour and feel and which does not generate unacceptable discomfort (tingling, tautness, redness), which may dissuade consumers from using this composition.

The emulsions according to the invention generally have a viscosity ranging from 0.5 Pa·s to 10 Pa·s, preferably from 1 Pa·s to 8 Pa·s and more preferably from 2 Pa·s to 6 Pa·s measured by means of a rheometer of Rheomat RM 180 type, the measurements being carried out at 24 h at 25° C. for 10 minutes at a shear rate of 200 s⁻¹ using spindles 3 or 4.

### Liposoluble Organic Screening Agents

The liposoluble organic UV-screening agents are chosen in particular from cinnamic derivatives; anthranilates; salicylic derivatives; dibenzoylmethane derivatives; camphor derivatives; benzophenone derivatives; β, β-diphenylacrylate derivatives; triazine derivatives; benzotriazole derivatives; benzalmalonate derivatives, in particular those mentioned in patent U.S. Pat. No. 5 624 663; imidazolines; p-aminobenzoic acid (PABA) derivatives; benzoxazole derivatives as described in patent applications EP 0 832 642, EP 1 027 883, EP 1 300 137 and DE 101 62 844; screening polymers and screening silicones such as those described in particular in patent application WO 93/04665; α-alkylstyrene-based dimers, such as those described in patent application DE 198 55 649; 4,4-diarylbutadienes such as those described in applications EP 0 967 200, DE 197 46 654, DE 197 55 649, EP-A-1 008 586, EP 1 133 980 and EP 133 981; merocyanins such as those described in patent U.S. Pat. No. 4 195 999, application WO 2004/006878, applications WO 2008/090066, WO 2011/113718 and WO 2009/027258, and documents IP COM JOURNAL No. 000179675D published on 23 Feb. 2009, IP COM JOURNAL No. 000182396D published on 29 Apr. 2009, IP COM JOURNAL No. 000189542D published on 12 Nov. 2009, IP COM Journal No. IPCOM000011179D published on Apr. 3, 2004 and mixtures thereof.

As examples of liposoluble organic UV-screening agents, mention may be made of those denoted hereinbelow under their INCI name:

Dibenzoylmethane Derivative

Butylmethoxydibenzoylmethane or avobenzone sold in particular under the trade name Parsol 1789 by the company DSM Nutritional Products;

para-Aminobenzoic Acid Derivatives:

Ethyl PABA,

Ethyl dihydroxypropyl PABA,

Ethylhexyl dimethyl PABA sold in particular under the name Escalol 507 by ISP;

Salicylic Derivatives:

Homosalate sold in particular under the name Eusolex HMS by Rona/EM Industries,

Ethylhexyl salicylate sold in particular under the name Neo Heliopan OS by Symrise;

Cinnamic Derivatives:

Ethylhexyl methoxycinnamate sold in particular under the trade name Parsol MCX by DSM Nutritional Products,

Isopropyl methoxycinnamate,

Isoamyl methoxycinnamate sold in particular under the trade name Neo Heliopan E 1000 by Symrise,

Cinoxate,

Diisopropyl methylcinnamate;

β, β-Diphenylacrylate Derivatives:

Octocrylene sold in particular under the trade name Uvinul N539 by BASF,

Etocrylene sold in particular under the trade name Uvinul N35 by BASF;

Benzophenone Derivatives:

Benzophenone-1 sold in particular under the trade name Uvinul 400 by BASF,

Benzophenone-2 sold in particular under the trade name Uvinul D50 by BASF,

Benzophenone-3 or oxybenzone sold in particular under the trade name Uvinul M40 by BASF,

Benzophenone-6 sold in particular under the trade name Helisorb 11 by Norquay,

Benzophenone-8 sold in particular under the trade name Spectra-Sorb UV-24 by American Cyanamid,

Benzophenone-12,

N-hexyl 2-(4-diethylamino-2-hydroxybenzoyl)benzoate sold in particular under the trade name Uvinul A+, such as Uvinul A+Granular, or in the form of a mixture with octyl methoxycinnamate in particular under the trade name Uvinul A+B by BASF;

Benzylidenecamphor Derivatives:

3-Benzylidenecamphor sold in particular under the name Mexoryl SD by Chimex,

4-Methylbenzylidenecamphor sold in particular under the name Eusolex 6300 by Merck,

Polyacrylamidomethylbenzylidenecamphor sold in particular under the name Mexoryl SW by Chimex;

Phenylbenzotriazole Derivatives:

Drometrizole trisiloxane sold in particular under the name Silatrizole by Rhodia Chimie or manufactured by Chimex under the trade name Meroxyl XL®.

Triazine Derivatives:

Bis(ethylhexyloxyphenol)methoxyphenyltriazine sold in particular under the trade name Tinosorb S by BASF,

Ethylhexyl triazone sold in particular under the trade name Uvinul T150 by BASF,

Diethylhexyl butamido triazone sold in particular under the trade name Uvasorb HEB by Sigma 3V,

silicone triazines substituted with two aminobenzoate groups, as described in patent EP 0 841 341, in particular 2,4-bis(n-butyl 4′-aminobenzalmalonate)-6-[(3-{1,3,3,3-tetramethyl-1-[(trimethylsilyl)oxy]disiloxanyl}propyl)amino]-s-triazine;

Anthranilic Derivatives:

Menthyl anthranilate sold in particular under the trade name Neo Heliopan MA by Symrise;

Imidazoline Derivatives:

Ethylhexyl dimethoxybenzylidene dioxoimidazoline propionate;

Benzalmalonate Derivatives:

Dineopentyl 4′-methoxybenzalmalonate,

Polyorganosiloxane comprising benzalmalonate functions, for instance Polysilicone-15 sold in particular under the trade name Parsol SLX by DSM;

4,4-Diarylbutadiene Derivatives:

1,1-Dicarboxy(2,2′-dimethylpropyl)-4,4-diphenylbutadiene;

Benzoxazole Derivatives:

2,4-Bis[5-(1,-dimethylpropyl)benzoxazol-2-yl-(4-phenyl)imino]-6-(2-ethylhexyl)imino-1,3,5-triazine sold in particular under the name Uvasorb K2A by Sigma 3V,

and mixtures thereof;

Merocyanin Derivatives:

Octyl 5-N,N-diethylam ino-2-phenylsulfonyl-2,4-pentadienoate; and mixtures thereof;

The preferential liposoluble organic screening agents are chosen from:

Butylmethoxydibenzoylmethane,

Ethylhexyl methoxycinnamate,

Ethylhexyl salicylate,

Homosalate,

Octocrylene,

Benzophenone-3,

n-Hexyl 2-(4-diethylamino-2-hydroxybenzoyl)benzoate,

4-Methylbenzylidenecamphor,

Bis(ethylhexyloxyphenyl)methoxyphenyltriazine,

Ethylhexyl triazone,

Diethylhexyl butamido triazone,

2,4,6-Tris(dineopentyl 4′-am inobenzalmalonate)-s-triazine,

2,4,6-Tris(diisobutyl 4′-am inobenzalmalonate)-s-triazine,

2,4-Bis(dineopentyl 4′-am inobenzalmalonate)-6-(n-butyl 4′-am inobenzoate)-s-triazine,

Drometrizole trisiloxane,

Polysilicone-15,

1,1-Dicarboxy(2,2′-dimethylpropyl)-4,4-diphenylbutadiene,

2,4-Bis[5-(1,1-dimethylpropyl)benzoxazol-2-yl-(4-phenyl)imino]-6-(2-ethylhexyl)imino-1,3,5-triazine,

and mixtures thereof.

The preferential liposoluble organic screening agents are more particularly chosen from:

Butylmethoxydibenzoylmethane,

Octocrylene,

Ethylhexyl salicylate,

n-Hexyl 2-(4-diethylamino-2-hydroxybenzoyl)benzoate,

Bis(ethylhexyloxyphenol)methoxyphenyltriazine,

Ethylhexyl triazone,

Diethylhexyl butamido triazone,

Drometrizole trisiloxane, and mixtures thereof.

The liposoluble organic UV-screening agent(s) are preferably present in the compositions according to the invention in a content ranging from 0.1% to 30% by weight and preferably from 0.1% to 25% by weight, and more preferably from 0.5% to 20% by weight relative to the total weight of the composition.

### Mixture of Nonionic Surfactants

The mixture of nonionic surfactants in accordance with the invention comprises:
(i) at least one alkoxylated fatty ester, and
(ii) at least one fatty ester of glycerol.

a) Alkoxylated Fatty Esters

The alkoxylated fatty esters in accordance with the invention are preferably esters formed from 1 to 100 ethylene oxide units and from at least one fatty acid chain comprising from 16 to 22 carbon atoms, preferably formed from 2 to 20 ethylene oxide units and from at least one fatty acid chain comprising from 16 to 22 carbon atoms. The fatty chain of the esters may be chosen in particular from stearate, behenate, arachidate and palmitate units, and mixtures thereof. Examples of alkoxylated fatty esters that may be mentioned include the stearic acid ester comprising 8 ethylene oxide units, such as the product sold under the name Myrj S8-SO-(MV)® (INCI name: PEG-8 stearate) by the company Croda, and also the behenic acid ester comprising 8 ethylene oxide units (INCI name: PEG-8 behenate), such as the product sold under the name Compritol HD5 ATO® by the company Gattefosse.

b) Fatty Esters of Glycerol

The fatty esters of glycerol in accordance with the invention may be chosen in particular from the group comprising the esters formed from at least one acid comprising a saturated linear alkyl chain containing from 16 to 22 carbon atoms and from 1 to 20 glycerol units, preferably from 1 to 12 glycerol units. One or more of these fatty esters of glycerol may be used in the emulsion of the invention.

These esters may be chosen in particular from stearates, behenates, arachidates and palm itates, and mixtures thereof. Stearates and palm itates are preferably used.

As examples of surfactants that may be used in the emulsion of the invention, mention may be made of decaglyceryl monostearate, distearate, tristearate and pentastearate (CTFA names: polyglyceryl-10 stearate, polyglyceryl-10 distearate, polyglyceryl-10 tristearate and polyglyceryl-10 pentastearate), such as the products sold under the respective names Nikkol Decaglyn 1-S®, 2-S®, 3-S® and 5-S® by the company Nikko, and diglyceryl monostearate (CTFA name: polyglyceryl-2 stearate), such as the product sold by the company Nikko under the name Nikkol DGMS®, and more particularly still polyglyceryl-2 stearate.

Use will be made more particularly of a mixture of PEG-8 stearate and polyglyceryl-2 stearate.

The amount of the mixture of nonionic surfactant of the invention may range, for example, from 0.2% to 15% by weight and preferably from 1% to 8% by weight relative to the total weight of the emulsion.

The weight ratio of the amount of oily phase to the total amount of ionic surfactants and anionic amphiphilic lipid(s) ranges from 2 to 20 and preferably from 3 to 10.

The term “amount of oily phase” here is intended to mean the total amount of the lipophilic constituents of this phase (fatty substances, liposoluble organic UV-screening agents) with the exception of the nonionic surfactants and the anionic amphiphilic lipid(s).

### Anionic Amphiphilic Lipids

The anionic amphiphilic lipids may be more particularly chosen from the group formed by:

alkali metal salts of dicetyl and dimyristyl phosphate;

alkali metal salts of cholesterol sulfate;

alkali metal salts of cholesterol phosphate;

lipoamino acids and salts thereof such as monosodium and disodium acylglutamates, for instance the disodium salt of N-stearoyl-L-glutamic acid (INCI name: Disodium Stearoyl Glutamate) sold under the name Amisoft HS21P® by the company Ajinomoto;

the sodium salts of phosphatidic acid;

phospholipids.

The alkylsulfonic derivatives may be more particularly chosen from alkylsulfonic derivatives of formula (I):

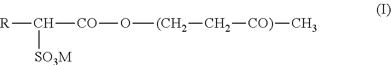

in which R represents an alkyl radical comprising from 16 to 22 carbon atoms, in particular the C₁₆H₃₃ and C₁₈H₃₇ radicals, taken as a mixture or separately, and M is an alkali metal such as sodium.

According to a preferred embodiment of the invention, a lipoamino acid is used as anionic amphiphilic lipid, and more particularly monosodium and disodium acylglutamates, such as the disodium salt of N-stearoyl-L-glutamic acid (INCI name: Disodium Stearoyl Glutamate) sold under the name Amisoft HS21P® by the company Ajinomoto.

The anionic amphiphilic lipids may be introduced into either of the phases of the emulsion. When they are present in the emulsion of the invention, they may be used in concentrations preferably ranging from 0.01% to 5% by weight and more particularly from 0.25% to 1% by weight relative to the total weight of the emulsion.

### Oily Phase

The oil globules of the emulsions of the invention have a number-average size of less than or equal to 200.0 nm, and preferably ranging from 50.0 to 200.0 nm and more particularly from 80 to 120 nm. The reduction in the size of the globules allows the penetration of active agents into the superficial layers of the skin to be promoted (carrier effect). The particle size of the oily globules is generally measured using a Brookhaven Instrument Corp particle size analyzer: 90 Plus Particle size analyzer.

The emulsion according to the invention comprises preferably at least one oil of molecular weight greater than 400 g/mol.

“Molecular weight” is intended to mean the ratio between the weight of a molecule and the atomic mass unit (amu) which is equivalent to 1/12 of the mass of a carbon 12 atom.

The oils may be chosen from oils of plant origin, mineral oils, synthetic oils and silicone oils, and mixtures thereof. As oils of this type, examples that may be mentioned include isocetyl palmitate, isocetyl stearate, avocado oil, soya bean oil, and more particularly soya bean oil.

Moreover, the oily phase may optionally contain other oils and in particular oils having a molecular weight of lower than 400.0 g/mol. These oils are also chosen from plant oils, mineral oils, synthetic oils and silicone oils. As an example of oils of molecular weight lower than 400.0 g/mol, mention may be made of isododecane, isohexadecane, volatile silicone oils, isopropyl myristate, isopropyl palm itate and C₁₁-C₁₃ isoparaffin.

The oily phase may also contain fatty substances other than the oils indicated hereinabove, such as fatty alcohols such as stearyl, cetyl and behenyl alcohols, fatty acids such as stearic, palmitic and behenic acids, fluoro oils, waxes, gums and mixtures thereof.

The emulsions in accordance with the invention comprise an amount of oily phase preferably ranging from 10% to 50% and better still from 20% to 40% by weight relative to the total weight of the emulsion.

### Aqueous Phase

The composition of the invention comprises an aqueous phase.

The aqueous phase contains water and optionally one or more other water-soluble or water-miscible organic solvents.

An aqueous phase that is suitable for use in the invention may comprise, for example, a water chosen from a natural spring water, such as water from La Roche-Posay, water from Vittel or waters from Vichy, or a floral water.

The water-soluble or water-miscible solvents that are suitable for use in the invention comprise diols or polyols, such as ethylene glycol, 1,2-propylene glycol, 1,3-butylene glycol, hexylene glycol, diethylene glycol, dipropylene glycol, 2-ethoxyethanol, diethylene glycol monomethyl ether, triethylene glycol monomethyl ether, glycerol and sorbitol, caprylyl glycol, pentylene glycol and mixtures thereof.

In the context of the present invention the term “polyol” is intended to mean an organic compound comprising at least 3 hydroxyl functions and typically from 3 to 10 hydroxyl functions.

According to one particular embodiment, the aqueous phase of the composition of the invention comprises a mixture of at least one diol and at least one polyol.

According to one particular embodiment, it is possible to use more particularly propylene glycol, glycerol, caprylyl glycol, pentylene glycol and mixtures thereof.

According to one particular form of the invention, the overall aqueous phase, including all the hydrophilic substances of the composition which are capable of being dissolved in this same phase, represents from 10% to 90% by weight and preferably from 30% to 60% by weight relative to the total weight of the composition.

According to one particular form of the invention, the emulsion comprises:

a) a mixture of nonionic surfactants comprising
(i) at least one alkoxylated fatty ester formed from 1 to 100 ethylene oxide units and from at least one fatty acid chain comprising from 16 to 22 carbon atoms, and more particularly PEG-8 stearate;
(ii) at least one ester of fatty acid and of glycerol formed from at least one acid comprising a saturated linear alkyl chain comprising from 16 to 22 carbon atoms and from 1 to 10 glycerol units, and more particularly polyglyceryl-2 stearate, and

b) at least one ionic amphiphilic lipid chosen from monosodium and disodium acylglutamates, and more particularly the disodium salt of N-stearoyl-L-glutamic acid;

c) at least one oil chosen frrom isocetyl palmitate, isocetyl stearate, avocado oil, soya bean oil, and more particularly soya bean oil.

### Additives

The emulsions defined hereinabove may be used in any field in which this type of composition is of use. They may in particular constitute compositions for topical use and in particular cosmetic or dermatological compositions.

The compositions in accordance with the present invention may be in the form of a more or less smooth cream or a gel-cream, and may also comprise standard cosmetic adjuvants chosen in particular from ionic or nonionic, hydrophilic or lipophilic thickeners, demulcents, humectants, antioxidants, stabilizers, emollients, silicones, antifoams, fragrances, preserving agents, lipophilic or hydrophilic active agents, dyes, polymers, propellants, or any other ingredient commonly used in the cosmetics and/or dermatological field.

Hydrophilic thickeners that may be mentioned include carboxyvinyl polymers, such as Carbopols (Carbomers) and the Pemulens® (acrylate/C10-C30 alkyl acrylate copolymer); polyacrylamides, for instance the crosslinked copolymers sold under the names Sepigel 305® (CTFA name: polyacrylamide/C13-14 isoparaffin/laureth 7) or Simulgel 600® (CTFA name: acrylamide/sodium acryloyldimethyltaurate copolymer/isohexadecane/polysorbate 80) by the company SEPPIC; 2-acrylamido-2-methylpropanesulfonic acid polymers and copolymers, optionally crosslinked and/or neutralized, for instance poly(2-acrylamido-2-methylpropanesulfonic acid) sold by the company Hoechst under the trade name Hostacerin AMPS® (CTFA name: ammonium polyacryloyldimethyl taurate) or Simulgel 800® sold by the company SEPPIC (CTFA name: sodium polyacryolyldimethyltaurate/polysorbate 80/sorbitan oleate); copolymers of 2-acrylamido-2-methylpropanesulfonic acid and of hydroxyethyl acrylate, for instance Simulgel NS and Sepinov EMT 10® sold by the company SEPPIC; cellulose derivatives such as hydroxyethylcellulose; polysaccharides and in particular gums such as xanthan gum; and mixtures thereof.

Among the active agents, mention may be made of:

vitamins (A, C, E, K, PP, etc.) and derivatives or precursors thereof, alone or as mixtures;

agents for combating pollution and/or free-radical scavengers;

depigmenting agents and/or propigmenting agents;

calmatives;

tensioning agents;

keratolytic agents;

desquamating agents;

moisturizing agents;

anti-wrinkle agents.

Needless to say, those skilled in the art will take care to choose the abovementioned optional additional compound(s) and/or the amounts thereof so that the advantageous properties intrinsically attached to the emulsions in accordance with the invention are not, or not substantially, detrimentally affected by the envisaged addition(s).

Another subject of the invention is a process for preparing an emulsion as defined hereinabove, this process consisting in mixing the aqueous phase and the oily phase comprising the lipophilic organic UV-screening agent(s) with vigorous stirring, at a temperature ranging from 10 to 85° C., then in homogenizing at a pressure lower than 1000.0 bar and preferably ranging from 300 to 700 barA.

Preferably, the emulsion is subjected to shearing ranging from 2×10⁶ s⁻¹ to 5×10⁸ s⁻¹, and better still from 1×10⁸ S⁻¹ to 3×10⁸ s⁻¹.

The emulsion of the invention may for example be used for caring for and treating the skin, the face and the body.

Another subject of the present invention consists of the use of the emulsions according to the invention as defined above for the manufacture of products for cosmetic treatment, in particular products for caring for the skin and anti-sun products, and more particularly anti-sun products.

The cosmetic compositions according to the invention may be used, for example, as care products and/or anti-sun protection products for the face and/or the body.

The examples that follow will allow the invention to be understood more clearly, without, however, being limiting in nature. The amounts shown are as % by weight.

| | Ex. 1 | |
|---|---|---|
| | (outside the | Ex 2 |
| Ingredients (INCI name) | invention) | (invention) |
| BHT | 0.08 | — |
| Triethanolamine | 0.1 | — |
| Adenosine | 1.00 | — |
| Glycine Soya (soya bean) Oil | 2.5 | 4.5 |
| Isononyl Isononanoate | 4.4 | — |
| Cetyl Alcohol | 1.1 | 1.1 |
| Isocetyl Stearate | 2.2 | — |
| Ethylhexyl Salicylate, | 5.0 | 5.0 |
| Octocrylene | 7.0 | 7.0 |
| Butylmethoxydibenzoylmethane | 3.0 | 3.0 |
| Fragrance | 0.22 | — |
| Cyclopentasiloxane | 3.3 | — |
| Dimethicone | — | 4.0 |
| Butylene glycol | 7.0 | 7.0 |
| Denatured alcohol (96°) | 5.35 | — |
| C₁₂-C₁₅-Alkyl benzoate | — | 4.4 |
| Glycerol | 3.0 | 3.0 |
| Pentylene glycol | — | 3.0 |
| Capryl glycol | — | 0.3 |
| Stearic Acid | 0.66 | 0.66 |
| PEG-8 Stearate | 1.54 | 1.54 |
| Disodium Stearoyl Glutamate | 0.85 | 0.85 |
| Polyglyceryl-2 Stearate | 2.37 | 2.37 |
| Tocopheryl Acetate | qs | qs |
| Preservatives | qs | qs |
| Water | qs 100 | qs 100 |
| VISCOSITY at 24 h at 25° C. | 3.6 | 2.3 |
| (Pa.s after 10 minutes at spindle 4) | | |
| SIZE OF DROPLETS | 96 | 103 |
| (nm) | | |
| SPF | 10.7 ± 3 | 25.9 ± 3 |

Procedure for the compositions:

The emulsions were produced using a valve homogenizer of the Soavi type under a pressure of 500 bar. A pre-emulsion is produced at 65° C. with stirring using a

Moritz mixer, after introduction of the oily phase into the aqueous phase and after prior melting of the constituents of the aqueous and oily phases. The emulsion is then produced at 30° C., placing the pre-emulsion in the Soavi homogenizer at 500 bar until a satisfactory particle size is obtained.

The viscosity was measured using a rheometer of Rheomat RM 180 type, at 24 hours at 25° C. for 10 minutes at a shear rate of 200 s⁻¹ using spindles 3 or 4. The particle size of the oily globules is measured using a Brookhaven Instrument Corp particle size analyzer: 90 Plus Particle Size Analyzer. The protection factor (SPF) was measured according to the method ISO24444-2010.

It was observed that alcohol-free composition 2 of the invention gave a higher SPF than composition 1 comprising alcohol.

1. An oil-in-water emulsion with globules of oil, the number-average size of which is less than or equal to 200.0 nm, comprising:
a) a continuous aqueous phase,
b) an oily phase dispersed in said aqueous phase,
c) a mixture of nonionic surfactants comprising:
(i) at least one alkoxylated fatty ester, and
(ii) at least one fatty ester of glycerol,
d) at least one anionic amphiphilic lipid,
e) at least one liposoluble organic UV-screening agent,
wherein the emulsion does not comprise any monoalcohol comprising from 2 to 8 carbon atoms, and that the weight ratio of the amount of oily phase to the total amount of nonionic surfactants and anionic amphiphilic lipid(s) is from 2 to 20.

2. The emulsion according to claim 1, wherein the liposoluble organic UV-screening agent(s) are chosen from cinnamic derivatives; anthranilates; salicylic derivatives; dibenzoylmethane derivatives; camphor derivatives; benzophenone derivatives; β, β-diphenylacrylate derivatives; triazine derivatives; benzotriazole derivatives; benzalmalonate derivatives; imidazolines; p-aminobenzoic acid (PABA) derivatives; benzoxazole derivatives; screening polymers and screening silicones; α-alkyl styrene-based dimers; 4,4-diarylbutadienes; merocyanins; and mixtures thereof.

3. The emulsion according to claim 1, wherein the liposoluble UV-screening agent(s) are chosen from the following compounds:
Butylmethoxydibenzoylmethane,
Octocrylene,
Ethylhexyl salicylate,
n-Hexyl 2-(4-diethylamino-2-hydroxybenzoyl)benzoate,
Bis(ethylhexyloxyphenol)methoxyphenyltriazine,
Ethylhexyl triazone,
Diethylhexyl butamido triazone,
Drometrizole trisiloxane, and mixtures thereof.

4. The emulsion according to claim 1, in which the alkoxylated fatty ester is formed from 1 to 100 ethylene oxide units and from at least one fatty acid chain comprising from 16 to 22 carbon atoms.

5. The emulsion according to claim 1, in which the fatty ester of glycerol is formed from at least one acid comprising a saturated linear alkyl chain containing from 16 to 22 carbon atoms and from 1 to 20 glycerol units.

6. The emulsion according to claim 1, wherein the amount of the mixture of nonionic surfactants ranges from 0.2% to 15% by weight relative to the total weight of the emulsion.

7. The emulsion according to claim 1, wherein the weight ratio of the amount of oily phase to the total amount of nonionic surfactants and anionic amphiphilic lipid(s) ranges from 3 to 10.

8. The emulsion according to claim 1, wherein the ionic amphiphilic lipids are chosen from the group formed by:
alkali metal salts of dicetyl and dimyristyl phosphate;
alkali metal salts of cholesterol sulfate;
alkali metal salts of cholesterol phosphate;
lipoamino acid salts;
the sodium salts of phosphatidic acid;
phospholipids;
alkylsulfonic derivatives of formula (I): 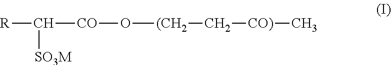
in which R represents C₁₆-C₂₂ alkyl radicals taken as a mixture or separately, and M is an alkali metal;
and mixtures thereof.

9. The emulsion according to claim 1, wherein the ionic amphiphilic lipids are chosen from monosodium and disodium acylglutamates.

10. The emulsion according to claim 1, wherein the amount of anionic amphiphilic lipid(s) ranges from 0.01% to 5% by weight relative to the total weight of the emulsion.

11. The emulsion according to characterized in that claim 1, wherein the oil globules have a number-average size ranging from 50 to 200 nm.

12. The emulsion according to claim 1, wherein the oily phase comprises at least one oil of molecular weight greater than 400 g/mol.

13. The emulsion according to claim 1, wherein the oily phase comprises at least one oil of molecular weight greater than 400 g/mol chosen from isocetyl palmitate, isocetyl stearate, avocado oil, soya bean oil.

14. The emulsion according to claim 1, wherein the amount of oily phase ranges from 10% to 50% relative to the total weight of the emulsion.

15. The emulsion according to claim 1, wherein the amount of aqueous phase ranges from 10% to 90% by weight relative to the total weight of the composition.

16. The emulsion according to claim 1, comprising:
a) a mixture of nonionic surfactants comprising:
(i) at least one alkoxylated fatty ester formed from 1 to 100 ethylene oxide units and from at least one fatty acid chain comprising from 16 to 22 carbon atoms,
(ii) at least one ester of fatty acid and of glycerol formed from at least one acid comprising a saturated linear alkyl chain comprising from 16 to 22 carbon atoms and from 1 to 20 glycerol units, and
b) at least one ionic amphiphilic lipid chosen from monosodium and disodium acylglutamates;
c) at least one oil chosen from isocetyl palmitate, isocetyl stearate, avocado oil, soya bean oil.

17. The emulsion according to claim 1, comprising a physiologically acceptable medium.

18. A process for preparing an emulsion according to claim 1, consisting in mixing the aqueous phase and the oily phase comprising the liposoluble organic UV-screening agent(s) with vigorous stirring, at a temperature ranging from 10 to 80° C., then in homogenizing at a pressure lower than 1000.0 bar.

19. The process according to claim 18, wherein the emulsion is subjected to shearing ranging from 2×10⁶ s⁻¹ to 5×10⁸ s⁻¹.

20. A cosmetic process for limiting the darkening of the skin and/or improving the colour and/or uniformity of the complexion, comprising the application, to the surface of the skin, of at least one composition as defined in claim 1.

21. A cosmetic process for preventing and/or treating the signs of ageing of a keratin material, comprising the application, to the surface of the keratin material, of a composition as defined in claim 1.

22. A cosmetic process for moisturizing a keratin material, comprising the application, to the surface of the keratin material, of a composition as defined above in claim 1.
